(19) United States
(12) Patent Application Publication
(10) Pub. No.: US 2022/0249099 A1
(43) Pub. Date: Aug. 11, 2022

(54) CATHETER-BASED MEDICAL DEVICE FOR OCCLUDING THE AORTIC FALSE LUMEN AFTER AN AORTIC DISSECTION

(71) Applicant: Grayson H. Wheatley, III, Nashville, TN (US)

(72) Inventor: Grayson H. Wheatley, III, Nashville, TN (US)

(21) Appl. No.: 17/248,739

(22) Filed: Feb. 5, 2021

(51) Int. Cl. A61B 17/12; A61B 17/00

(52) U.S. Cl. CPC A61B 17/12122; A61B 17/0057; A61B 17/12036; A61B 2017/1205; A61B 17/1215; A61B 17/12168; A61B 2017/00592; A61B 17/12145

(57) ABSTRACT

A catheter-based medical device for occluding the aortic false lumen after an aortic dissection. The medical device is embodied in a crescent-shaped catheter-based device made from a framework for retaining its unique shape, wherein the framework is integrated with a fabric adapted to accelerate thrombosis of blood flow and accelerate thrombosis of the false lumen at and proximal to the medical device. The crescent-shaped device may be adapted to be collapsed into a catheter-based delivery sheath.

### BACKGROUND OF THE INVENTION

The present invention relates to medical devices and, more particularly, a catheter-based medical device for occluding the aortic false lumen after an aortic dissection.

As with all other arteries, the wall of the aorta is made up of three layers, the intima, the media and the adventitia, wherein the intima is in direct contact with the blood inside the aorta. Aortic dissection results from a tear in the intima which allows blood to flow between the layers of the aortic wall, forcing the layers apart. Due to the high pressures in the aorta, blood enters the media at the point of the tear. The force of the blood entering the media causes the blood to travel through the media, creating a false lumen. Separating the false lumen from the true lumen is a layer of intimal tissue known as the intimal flap or aortic dissection septum.

Blood flow within the false lumen of the aorta following an aortic dissection increases the risk of aortic rupture and/or the development of an aortic aneurysm. In other words, there is an ongoing risk of death as a consequence of aortic rupture in individuals who continue to have persistent blood flow in their aortic false lumen. Even following successful open surgical repair or Thoracic Aortic Endovascular Repair (TEVAR) of an aortic dissection, the distinct possibility remains that blood flow can enter the residual false lumen adjacent to the repaired aorta from a distal fenestration (communication) of the aortic dissection septum. Blood entering the false lumen distal to the repaired aorta can perfuse the false lumen in a retrograde fashion, thus adversely impacting the success rate of the dissection repair.

Current catheter-based devices that are designed to occlude blood flow in a channel are circular in nature and so have limited conformability with a non-circular or non-oval (natural crescent shape) cross-sectional plane typical of the aortic false lumen, thereby limiting their ability to completely occlude blood flow in such a cross-section. Furthermore, placing a circular or oval-shaped device into the aortic false lumen frequently creates gutters immediately adjacent to the device due to the mismatch between the cross-sectional geometries of the occluding device and the aortic false lumen. Blood flow can persist into the aortic false lumen as a result of the residual gutters between current devices and the crescent-shaped nature of aortic false lumens, wherein such persistent blood flow can lead to aortic rupture over time.

As can be seen, there is a need for a catheter-based medical device for occluding the aortic false lumen after an aortic dissection to prevent future aortic deterioration and possible rupture. The present invention embodies a crescent-shaped, self-expanding medical device delivered through a delivery sheath using standard endovascular techniques. It is dimensioned and adapted to optimally conform to the cross-sectional nature of the aortic false lumen channel, eliminating any gaps between the occluding device and the aortic wall and septum of the aortic dissection.

The present invention would be used in conjunction with a TEVAR repair to successfully prevent retrograde filling of the false lumen in order to minimize the chances for subsequent aortic rupture or aneurysm formation. The present invention is dimensioned and adapted to completely fill the unique anatomy of the false lumen after stent-graft repair of an aortic dissection. The interventional placement of the occluding device of the present invention is disposed at a particular place of the aortic dissection and at a discrete level relative to the aorta. The particular place and discrete level are similar, but not the same. The “axis of deployment” is the centerline reconstruction down the center of the aorta. Anatomically, the aorta is not straight-up and down. The device will be deployed in a manner that is orthogonal to the centerline at a level commensurate with the end of the existing stent-graft (not shown) within the true lumen of the aortic dissection. It is the practitioner's judgment and discretion on how and where to deploy the device based on an individual patient's anatomy.

The present invention is adapted to be interventionally delivered as a catheter-based solution. The device is tethered to a detachable delivery wire, and the whole apparatus is delivered to its intended target area (the aortic false lumen) using a delivery sheath (not shown). Due to the “memory” elastic deformation properties of a nitinol metal mesh, the device can be retrieved and recaptured within the delivery sheath to allow for repositioning and optimal placement within the aortic false lumen.

### SUMMARY OF THE INVENTION

In one aspect of the present invention, a device for occluding an aortic false lumen, the device having a framework having a crescent shape having a volumetric depth, wherein the framework comprises a mesh made of a memory material with super-elastic deformation properties; a thrombotic fabric attached along an interior surface of the framework, wherein the thrombotic fabric accelerates thrombosis of blood flow by interacting with clotting factors in blood, wherein the memory material is nitinol, wherein the framework is collapsible, whereby the framework collapses into a catheter-based delivery sheath, wherein the crescent shape defined in part by a concave-convex region bounded by two circular arcs, wherein a first diameter of a first circular arc of the two circular arcs is between 22 mm and 46 mm, and wherein second diameter of a second circular arc of the two circular arcs is between 14 mm and 32 mm, and wherein the volumetric depth is between 16 mm and 26 mm.

In another aspect of the present invention, the device for occluding an aortic false lumen, the device further includes a spacing rod interconnecting the framework and another device, wherein the other device of claim 6 has a first diameter and second diameter than the framework, whereby two different discrete cross sections of the aortic false lumen are occluded.

These and other features, aspects and advantages of the present invention will become better understood with reference to the following drawings, description and claims.

### BRIEF DESCRIPTION OF THE DRAWINGS

FIG. 1 is a cross-section view of an exemplary embodiment of the present invention, illustrating how D1 and D2 are skew to differentiate these dimensions from the x and y axis;

FIG. 2 is a perspective view of an exemplary embodiment of the present invention; and

FIG. 3 is a perspective view of an exemplary embodiment of the present invention, shown in use, without a TEVAR device shown in the true lumen adjacent to the present invention, demonstrating that the present invention could also be deployed without a TEVAR device.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a crescent-shaped catheter-based device made from a framework for retaining its unique shape. The framework is a mesh (or “meshwork”) that is malleable and deformable but has a memory to return to its original state, wherein the meshwork is integrated with a fabric adapted to accelerate thrombosis of blood flow and accelerate thrombosis of the false lumen at and proximal to the medical device. The crescent-shaped device may be adapted to be collapsed into a catheter-based delivery sheath.

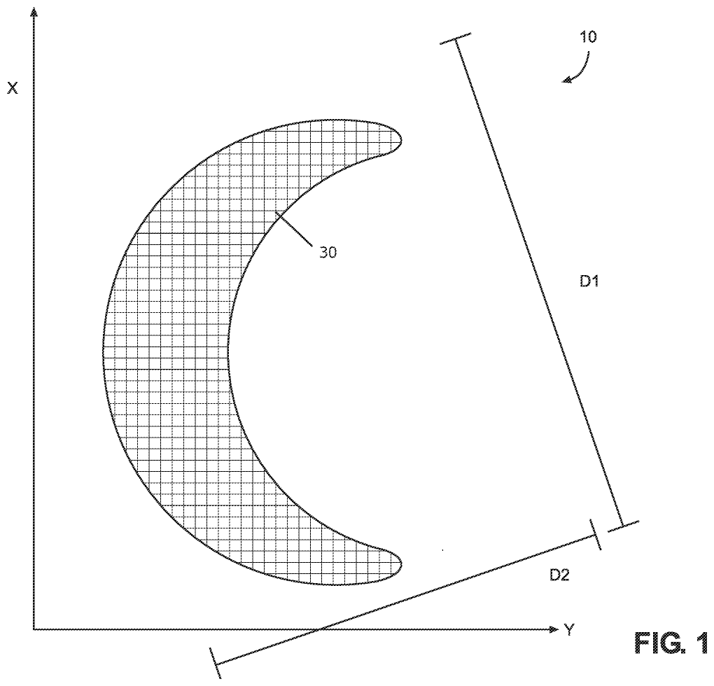

Referring now to FIGS. 1 through 3, the present invention may include a crescent-shaped catheter-based device 10. The crescent-shaped catheter-based device 10 may include a self-expanding three-dimensional meshwork 30 with operatively associated fabric creating a three-dimensional framed crescent shape device that is dimensioned and adapted to conform to and be coextensive with an inner cross section of a false lumen 14 of the aorta 16. The meshwork 30 may be constructed from a special alloy called “nitinol” or other memory material with super-elastic deformation properties, whereby the present invention always expands to its desired shape. When released from the delivery sheath, the present invention is a self-expanding plug.

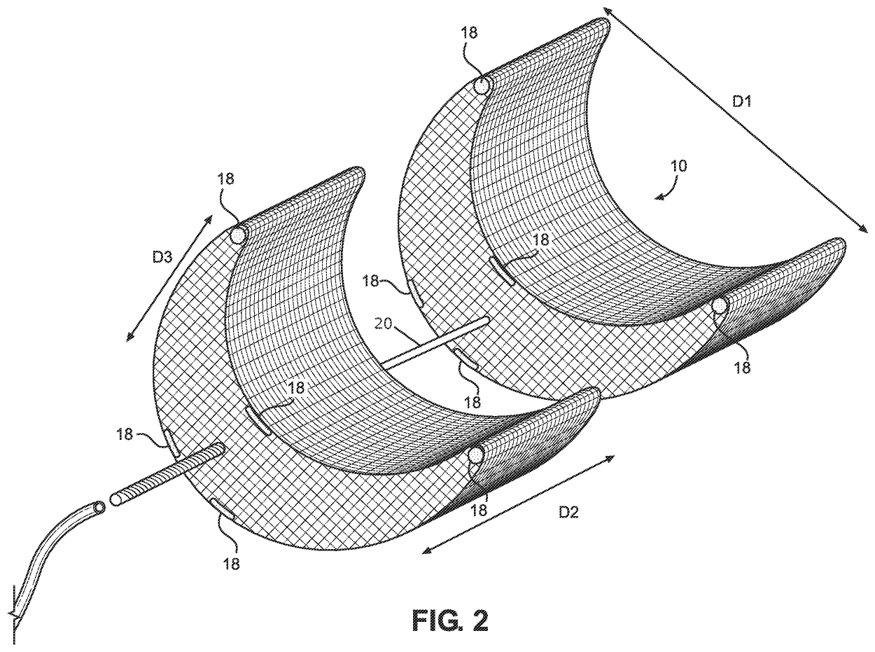

In plane geometry, the two-dimensional crescent shape is defined by the concave-convex region bounded by two circular arcs. According to FIG. 2, D1 is the diameter of one of the two circular arcs, while D3 is the diameter of the other of the two circular arcs. D2 is the volumetric depth of the three-dimensional present invention. The dimensions of D1, D2, and D3, as illustrated in the FIG. 2, may be as follows: D1 may range between 22 mm and 46 mm; D2 may range between 16 mm and 26 mm; and D3 may range between 14 mm and 32 mm.

A method of manufacturing the present invention may include the following. The three-dimensional meshwork 30 may be made from medical-grade nitinol metal woven into a three-dimensional mesh in the shape of a crescent using metallurgy and machines to retain its unique shape. The fabric may be affixed to and along an interior of the three-dimensional meshwork 30 through any sufficient medical-grade joining element.

The fabric has no impact on the expansion or shape of the device. The fabric is added to help create thrombosis (clot) of the blood which seals-up the meshwork 30 in order that the device can act as a “cork in a bottle”. The blood clots not by being absorbed by the fabric, but by interacting with clotting factors in the blood.

The fabric within the inside of the meshwork 30 could be like a sphere or elongated balloon or can take a range of different designs depending on the development the optimal form of the fabric during manufacturing. It will be necessary to have a range of different diameters and heights of three-dimensional meshwork 30 configurations in order to accommodate different aortic and false lumen diameters. The nature and design of the fabric relative to the nitinol mesh framework 30 can take a number of different forms (such as strands of fabric, complete covering or internal meshwork.

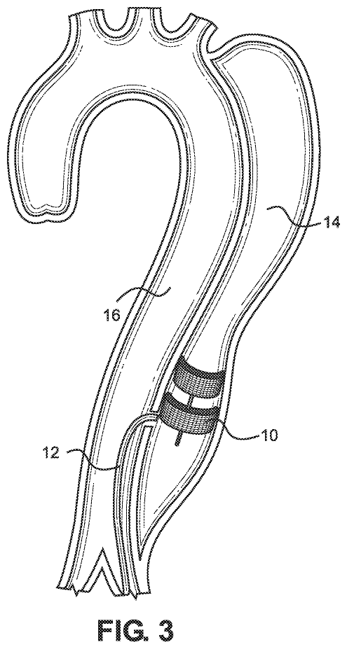

A method of using the present invention may include the following. The crescent-shaped catheter-based device 10 disclosed above may be provided, and due to the unique memory-like properties of nitinol, may need to be collapsed into a sheath for delivery via a catheter 12, as illustrated in FIG. 3. Using standard endovascular techniques and fluoroscopy, the delivery sheath containing the collapsed device 10 will be advanced through the aorta 16 (from a vascular access point) and deployed by releasing the device 10 and allowing the nitinol metal to expand into its crescent configuration at the desired location in the aortic false lumen 14.

In certain embodiments, two or more crescent-shaped devices 10 may be selectively spaced apart along a spacing rod 20 in contemplation of using (possibly) two differently dimensioned devices 10 for different discrete cross sections of the patient's false lumen 14, as illustrated in FIG. 3. Additionally, along a periphery of the crescent-shaped device 10 a plurality of radio opaque markers 18 may be disposed for facilitating the catheter-based process.

It should be understood, of course, that the foregoing relates to exemplary embodiments of the invention and that modifications may be made without departing from the spirit and scope of the invention as set forth in the following claims.

What is claimed is:

1. A device for occluding an aortic false lumen, the device comprising:
a framework having a crescent shape having a volumetric depth, wherein the framework comprises a mesh made of a memory material with super-elastic deformation properties.

2. The device of claim 1, further comprising a thrombotic fabric attached along an interior surface of the framework, wherein the thrombotic fabric accelerates thrombosis of blood flow by interacting with clotting factors in blood.

3. The device of claim 2, wherein the memory material is nitinol.

4. The device of claim 2, wherein the framework is collapsible,
whereby the framework collapses into a catheter-based delivery sheath.

5. The device of claim 4, wherein the crescent shape defined in part by a concave-convex region bounded by two circular arcs, wherein a first diameter of a first circular arc of the two circular arcs is between 22 mm and 46 mm, and wherein second diameter of a second circular arc of the two circular arcs is between 14 mm and 32 mm.

6. The device of claim 5, wherein the volumetric depth is between 16 mm and 26 mm.

7. The device of claim 6, further comprising a spacing rod interconnecting the framework and a framework of a second device.

8. The device of claim 7, wherein said second device has a first diameter and a second diameter that is different than the first diameter and the second diameter, respectively, of said device,
whereby two different discrete cross sections of the aortic false lumen are occluded.
